(19) United States
(12) Patent Application Publication
(10) Pub. No.: US 2023/0330322 A1
(43) Pub. Date: Oct. 19, 2023

(54) DEVICE FOR MOTORIZING THE MOVEMENTS OF THE PISTON OF A FLUID TANK USED IN THE MEDICAL ENVIRONMENT

(71) Applicant: APOLLINA, Alixan (FR)

(72) Inventors: Pierre DUTHOIT, Malissard (FR); Jeanne DELALANDE, Valence (FR); Baptiste LAGRANGE, Valence (FR)

(21) Appl. No.: 17/720,808

(22) Filed: Apr. 14, 2022

(51) Int. Cl. A61M 3/02

(52) U.S. Cl. CPC A61M 3/02; A61B 2017/00398

(57) ABSTRACT

A portable medical device (D) for using an interchangeable connecting tip is disclosed. The device includes an elongated body and can be carried and used with one hand. The body is configured at one end to removably receive said connecting tip, which includes a reservoir and a piston associated with a rod. The device also includes an actuator ensuring the movement of a first shaft acting on a second end of the rod. The reservoir can be an empty reservoir to be filled, and the first shaft is equipped to be temporarily fixed to the second end of the rod associated with the piston so as to, on the one hand, transmit by means of the first shaft, forward and reverse longitudinal movement of said piston so that the user can, fill and empty said reservoir.

### FIELD OF APPLICATION OF THE INVENTION

The present invention relates to the medical environment and in particular to adaptations allowing the piston of a fluid reservoir used in the medical environment to be set in motion.

### DESCRIPTION OF THE PRIOR ART

In the dental medical field, the objective of the treatment commonly called “devitalization” is to transform a pathological tooth into a healthy, asymptomatic and functional entity on the dental arch, this means ensuring its masticatory, manducatory (wedging, guiding and centering of the occlusion), phonetic and aesthetic functions.

To achieve this, the dental practitioner performs both chemical and mechanical disinfection of the root canal system. The dentist typically uses a rotating nickel-titanium instrument and irrigation solutions. In the dental medical field, pocket irrigation aims to cleanse plaque from the interdental (between teeth) and subgingival (under the gumline) regions of the mouth in order to prevent the colonization of harmful oral bacteria. This procedure is also used to deliver antibacterials to the subgingival areas. Its benefits are interdental cleaning, halitosis prevention, subgingival cleaning and application of antimicrobial agents.

In addition to root canalization and irrigation of periodontal pockets, the dental surgeon can perform other operations with the same tool:
Devitalization
Application of plaque developer
Application of caries developer
Application of dental cleaning products
Cleaning before prosthetic bonding or composite bonding
Saline irrigation during surgery
Application of platelet-rich plasma

While the irrigation syringe still has a place in the irrigation process during the procedure, a growing number of devices and equipment for activating solutions have recently emerged to complement the action of the irrigation syringe and optimize the effectiveness of trimming and disinfection.

This is the case of mechanized irrigation devices of the syringe pump type, which are increasingly replacing or combining with these syringes, technically surpassing them. These devices use syringes or piston tanks with a dedicated shape.

Nevertheless, whether they are manual or implemented by mechanical means, syringes or piston reservoirs present a major disadvantage related to their filling. Indeed, the filling is classically done manually, with two hands and requires a certain force due to the suction effect of the piston in the body of the syringe.

However, while there are a number of motorized mechanical solutions for emptying a syringe, the same cannot be said for filling syringes.

For example, as described in document FR3079407, there exists a dental electronic device for universal application with controlled thrust, which is made up of an elongated body inside which is positioned a battery that supplies an electric motor or geared motor for driving a thrust axis that is arranged to cooperate with a connecting tip from a range of connecting tips and that locks inside said elongated body.

Each connection tip comprises, on the one hand, fixation means for its retention inside said elongated body and, on the other hand, recognition means which are activated when the end-piece is connected and make it possible to initialize one of the control programs previously integrated into an electronic card housed inside said body and connected to a control button placed on the external face of the elongated body in order to control the electric motor or geared motor as a function of the determined control program of the end-piece, so as to control the latter.

According to one operating mode, one of the connection tips can be a piston tank whose thrust axis provides for the movement thereof for emptying purposes.

However, this type of device is not yet capable of enabling the filling of the said tank.

Another need that this type of device may have to meet is the heating of the product in the tank. Indeed, it has long been known that controlling the temperature of dental materials, before or during their use, has been of clinical benefit, especially for the application of dental composites, the injection of anesthetics or during endodontic irrigation.

### BRIEF DESCRIPTION OF THE INVENTION

Based on this observation, Applicant conducted research in order to:
Propose a motorized device being capable of filling a piston reservoir such as a syringe,
Improve the accessibility of the practitioner during the filling of a piston reservoir such as irrigation syringes in particular in the dental field which are used for example for the cleaning and the endocanalar disinfection during the devitalization procedures of the dental organs,
Make the procedure easy to perform by the practitioner with one hand.

This research has led to the design and production of a portable medical device for use with an interchangeable connection tip that can be used not only in dental applications but more generally in medical applications such as plastic surgery, veterinary or orthopedics among others. The invention can also be used to complement the functionalities of existing motorized devices.

The device of the invention comprises an elongated body carried and used with one hand,
said body being configured at one end to removably accommodate said connecting tip,
said connecting tip comprising a reservoir and a piston associated with a rod,
said rod comprising a first and a second end, respectively toward the front end and toward the rear end of the device, the first end being equipped with the piston moving in said reservoir,
said device motorizing the movement of said piston,
the device comprising an actuator ensuring the movement of a shaft acting on the second end of the rod,
said actuator being controlled by the user.

In accordance with the invention, this device is noteworthy in that the said reservoir is an empty reservoir to be filled and the first shaft is equipped to be fixed in a removable way to the second end of the rod associated with the piston so as to, on the one hand, transmit by means of the shaft a forward and backward translational movement to the second end of the rod associated with the piston so as to, on the one hand, transmit, by means of the first shaft, a forward and backward translational movement to said piston so that the user can, fill and empty said reservoir and, on the other hand, authorize the disassembly of the rod to allow the removal of the connection tip from the body.

In accordance with the objectives of the invention, this device allows the implementation of a filling operation of an interchangeable connecting tip such as a piston reservoir or a syringe with only one hand.

Indeed, the device of the invention drives in translation forward and backward along the axis of the piston.

For this purpose, according to the invention, the second end of the rod associated with the piston and the first shaft are connected together to allow transmission of movement forward and backward along the axis of the piston.

In addition, the second end of the piston and the first shaft can be disengaged to allow the tip to be changed, in particular the syringe tip if the latter is for single use.

According to a preferred but non-limiting embodiment, the device is a device used in a dental environment.

The piston reservoir is part of a first series of tips that can be actuated by the device.

According to one implementation of the invention, the device comprises a coupling module in helical connection with the first thrust shaft formed by a worm screw to be moved in translation back and forth when said screw is driven in rotation, said coupling module comprising a first fastening means intended to cooperate with a first series of connecting tips.

The use of a worm screw gear transmission makes it possible to use an electric motor with a motor-reduction module that provides the power necessary not only for pushing but also for suction.

According to a preferred but non-limiting embodiment, the electrical energy is provided by means of a battery accommodated in the body of the device. According to another preferred but non-limiting embodiment, this battery is induction charged by means of a coil also accommodated in the body of the device and recharging the battery once the device is placed on a support and recharging base.

According to another feature of the invention, the device also comprises a support and recharging base on which the body of the device can be positioned. This base can also be used to receive the tips.

According to another particularly advantageous feature of the invention, the coupling module comprises a second fastening means which, ensuring thrust only, is intended to cooperate with a thrust shaft cooperating with a second series of connection tips.

This plurality of fastening means brings additional functionalities in comparison with the already existing motorized devices which do not enable the filling of the tank and only provide translational thrust in one direction, limiting the number of possible functions.

According to a preferred but non-limiting embodiment, the coupling module comprises a second fastening means for releasably fastening the second push shaft.

The second shaft can also be included in the coupling module.

In a preferred but non-limiting embodiment, the second fastening means provides retention of a second shaft engaging with the second series of connection tips without interfering with the use of the first series of connection tips configured for this purpose.

Thus, another object of the present invention is to improve the drive and engagement of the thrust shaft of dental electronic devices with connecting tips already known in the prior art.

According to another particularly advantageous feature of the invention, the first and second fastening means of the coupling module are coaxial with the longitudinal axis of the worm screw.

According to another particularly advantageous feature of the invention, the coupling module comprises on its outer periphery a rotation blocking means so that its movement along the first shaft is a translational movement. This means blocks the rotation of the coupling module which, under the action of rotation of the worm screw, moves in translation in the body of the device.

According to another particularly advantageous feature of the invention, the second end of the piston is configured with a retaining bead in order to engage with the coupling module.

According to another particularly advantageous feature of the invention, the second end of the rod associated with the piston is configured with a plurality of slots in order to engage with the coupling module by being attached thereto by elastic deformation.

According to another particularly advantageous feature of the invention, the second end of the rod associated with the piston is configured with a hollow core flaring outwardly with a partially frustoconical inner surface so that the lamellae created by the slots have a thickness that reduces from their base to their end. This thickness decrease contributes to a better disengagement by giving more angular freedom to the lamella to better disengage from the coupling module.

Thus, according to another particularly advantageous feature of the invention, the coupling module, in its translational travel, couples to the second end of the rod associated with the piston once the connection tip is installed in the body in order to actuate it in both directions of translation.

According to another particularly advantageous feature of the invention, the second end of the rod associated with the piston further comprises a peripheral disengagement wedge that comes into contact at one end of the stroke of the coupling module with a disengagement stop allowing it to deform elastically so that its retaining bead is no longer retained in the coupling module. Thus, the coupling and uncoupling of the connection tip formed by the piston tank is carried out automatically.

Thus, according to a particularly advantageous feature of the invention, the dispenser comprises a button for controlling the filling operations of the reservoir that is advantageously arranged at the opposite end from that of the body accommodating the tip. The positioning of the said button, opposite the application zone, being in the medical field the mouth of the patient, makes it possible to leave the said zone free, so that the end of the syringe can easily enter a container filled with the irrigation solution.

Indeed, when, for reasons of hygiene, the dental practitioner pours the solution into disposable containers such as plastic cups, the use of the device with one hand is not hindered by the presence of his or her fingers, which are then at the opposite end of the container for filling. The buttons controlling the filling cannot be positioned on the front of the device since the practitioner's fingers would come into contact with the containers, thus preventing accessibility and filling. Nevertheless, the dispenser comprises one or more of the buttons involved in the use of the device for operations other than filling that are placed as close as possible to the end where the tip is located.

One technical solution incorporated into certain implementations of the device makes it easy and precise to fill a medical fluid reservoir such as an irrigation syringe in the dental field. This solution also improves accessibility and ergonomics, particularly during the irrigation operation.

According to another feature of the invention, the device comprises a means for heating the tip. Heating dental composite resins improves their mechanical properties and thus increases their hardness, adhesion, and degree of conversion. Heating and maintaining the tips at the ideal temperature reduces the sensation of injection, related to a sudden change in temperature and thus improves patient comfort. Finally, heating endodontic solutions increases their solvent power, increases the dissolution of organic and mineral materials by neutralizing microorganisms, and thus improves the irrigation treatment.

This heating of the tip can be implemented in several ways.

For example, according to a preferred embodiment, the heating means is implemented by one of the following means:
electrical resistance,
chemical reaction,
heating light,

According to another feature of the invention, the means for heating the connection tip is integrated or associated with one of the following sub-assemblies:
The device's body
The connection tip
The recharging base

The fundamental concepts of the invention having been set forth above in their most elementary form, other details and characteristics will become clearer upon reading the following description and in view of the appended drawings, which show, by way of non-limiting examples, embodiments of a device in accordance with the invention.

### BRIEF DESCRIPTION OF THE DRAWINGS

FIG. 1 is a schematic drawing of an external top view of an embodiment of a device according to the invention;

FIG. 2 is a schematic drawing of an exterior side view of the device of FIG. 1;

FIG. 3 is a schematic drawing of an exterior perspective view of the device of FIG. 1;

FIG. 4 is a schematic drawing illustrating the use of the device of FIG. 1 in a filling operation;

FIG. 5 is a schematic drawing illustrating the use of another embodiment of the device during a filling operation;

FIG. 6 is a schematic drawing of a top cross-sectional view of the device of FIG. 1 in the unsecured position;

FIG. 7 is a schematic drawing of a cross-sectional side view of the device of FIG. 1 in the unsecured position;

Previously shown in unsecured—this would be preferable otherwise this phase would not be shown

FIG. 8 is a schematic drawing of an exploded perspective view of a tank to be filled with its piston;

FIG. 9 is a schematic drawing of a side sectional view of said tank accommodating said piston;

FIG. 10, FIG. 11 and FIG. 12 are schematic drawings of front, cross-sectional side and perspective views of the coupling means;

FIG. 13 is a schematic drawing of a perspective view of a push shaft associated with the coupling means to use a second series of connection tips;

FIG. 14 is a schematic drawing of a perspective view of the motor support,

FIG. 15 is a schematic drawing of a cross-sectional top view of the motor support and the elements it houses;

FIG. 16, FIG. 17, FIG. 18, FIG. 19 and FIG. 20 are schematic drawings of cross-sectional views of the device adopting the following respective positions:
securing the syringe tip.
filling the syringe.
use.
disengaging of the syringe plunger,
disengaging the syringe.

FIG. 21 is a schematic drawing of a cross-sectional side view of the device of FIG. 1 accommodating a tip of a second series.

FIG. 22 is a schematic drawing of a side view in section of the device of FIG. 1 housing a tip of a second series associated with a heating means,

FIG. 23 is a schematic drawing of a cross-sectional side view of another of the device of the invention in which a heating means is integrated into the body,

FIG. 24 is a schematic drawing of an exterior perspective view of the device of FIG. 1 arranged on another embodiment of a support and recharge base accommodating the tips and the means for heating them.

FIG. 25 is a schematic drawing of a side view in section of the actuator causing the setting in motion of the first shaft.

### DESCRIPTION OF THE PREFERRED MODES OF IMPLEMENTATION

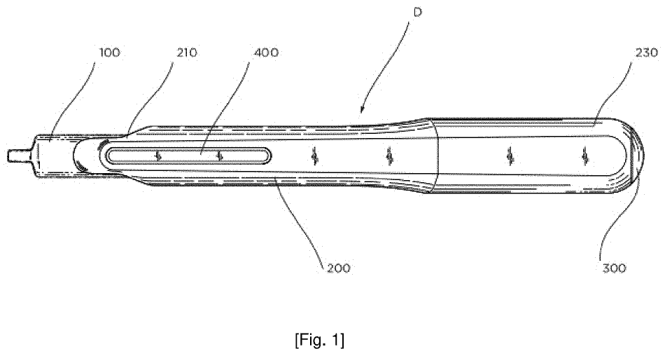

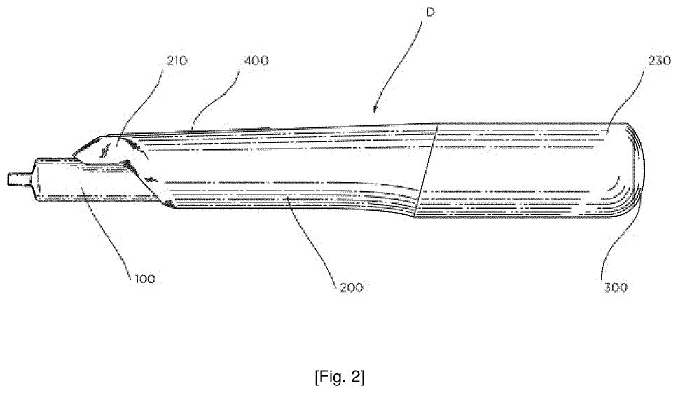

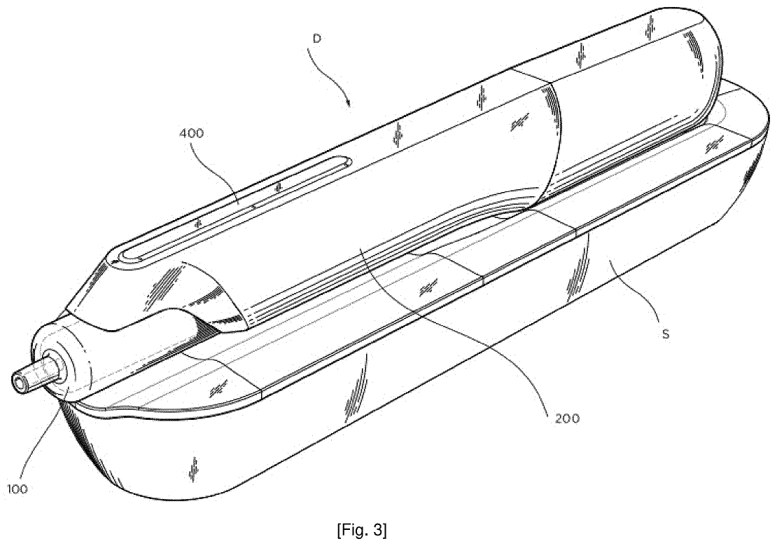

As illustrated in FIGS. 1, 2, 3, 4, 6 and 7, the portable medical device referenced D as a whole accommodates and utilizes a connecting tip such as an interchangeable piston reservoir 100.

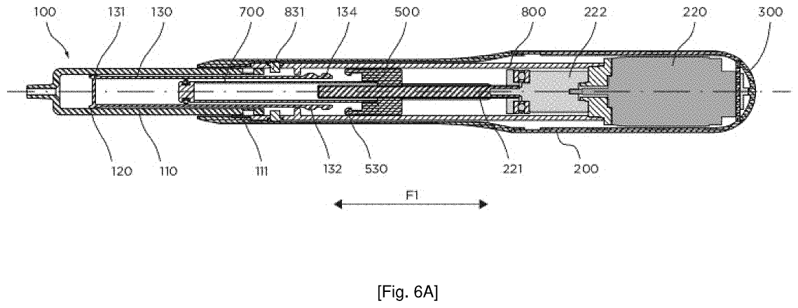

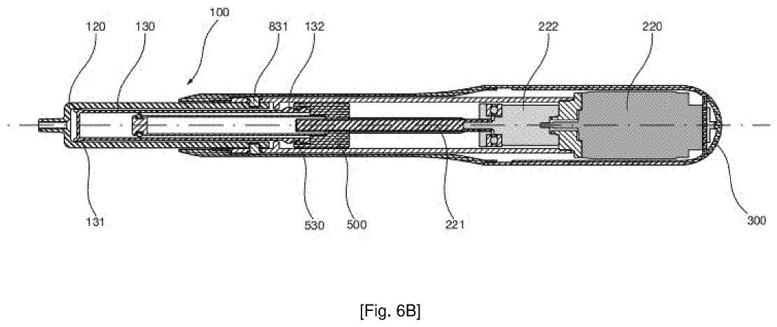

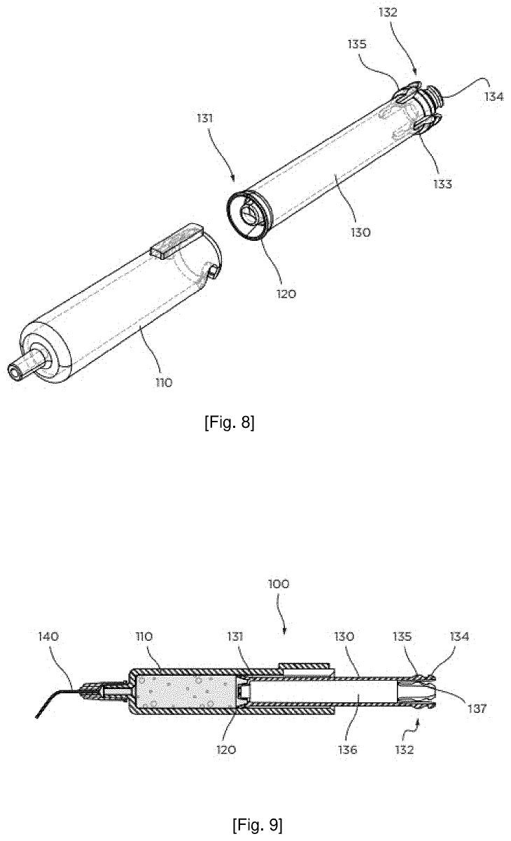

This device D comprises a body 200 of elongated shape such as a large diameter pen that can be carried and used with one hand. This body 200 is configured at a first end 210 to be open and to removably receive interchangeable tips, including said reservoir 100, which is inserted into the body through said open end 210. This device D ensures the motorization of the movement of the piston rod 130 (cf. FIG. 8) and thus comprises, housed in the body 200, a motor 220 (cf. FIG. 6).

As illustrated in FIGS. 6 to 9, the connecting end or piston reservoir 100 comprises:
a reservoir 110,
a piston 120 moving within the reservoir 110,
a piston rod 130 comprising a first end 131 and a second end 132, the first end 131 of the rod 130 being preformed to form the piston 120 moving within said reservoir 110.

According to another embodiment not illustrated, the piston is constituted by a flexible element (for example made of silicone) added to connect with the first end of the piston rod.

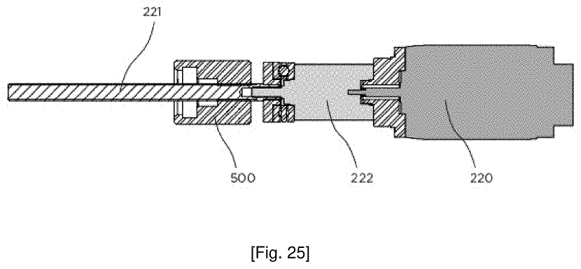

The motor 220 ensures the rotational movement of a shaft 221 (see FIGS. 6, 7 and 25) acting on the second end 132 of the piston 130. It is supplied with electricity by at least one battery A which stores the electrical energy which, according to the preferred but non-limiting embodiment illustrated, could be transferred by induction by means of a coil B when positioning the device D on a charging base S (FIG. 3).

This motor 200 is controlled by the user by means of at least one control button 300. This control button 300 is the control button for the filling operations of the reservoir 100 operated by the same hand that is already handling the device D and is advantageously disposed on the body 200 at the opposite end 230 of the first end 210 receiving the piston reservoir 100.

It can be associated with other buttons such as the button 400 which is located on the body 200 at the first end 210. This button 400 is preferably used once the tank 100 is filled.

As illustrated, this control button 300 which is, according to a preferred mode of a button controlling the retraction, closes the hollow core of the body 200 and is arranged so that its movement is in the longitudinal axis of the body 200.

Another embodiment is illustrated in FIG. 5, where the knob 300′ is arranged transversely to the longitudinal axis of the body 200′ of the device D′.

These two proposed positions of the control button allow the user to use his thumb in a comfortable and ergonomic position similar to the use of a manual pipette.

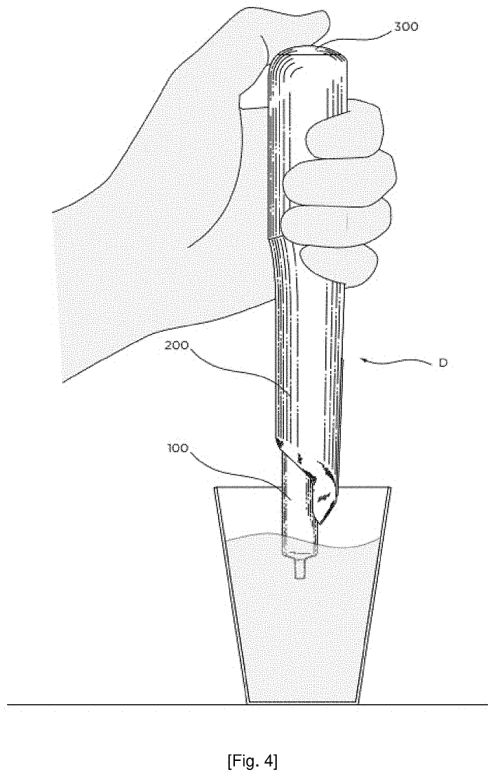

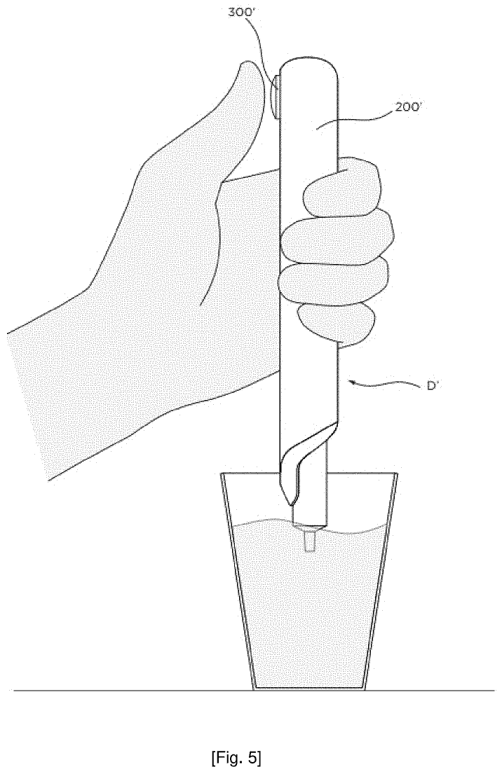

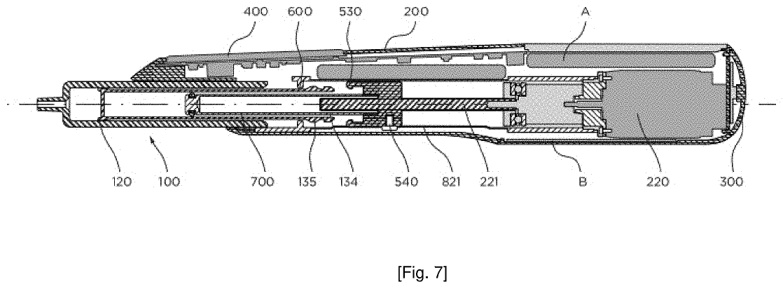

As illustrated by FIGS. 6 and 7, said reservoir 100 is an empty reservoir to be filled with the piston 120 at the end that can be equipped with the needle 140 (angled in FIG. 9). The filling operation is preferably performed without a needle or nozzle, as illustrated in FIGS. 4 and 5, so as not to require too much effort for suction.

The drive shaft 221 is equipped to be attached in a detachable way to the second end 132 of the piston rod 130 in order to:
on the one hand, to transmit by means of the shaft 221 a forward and return translation movement (double arrow F1) to said piston 120 so that the user can, by action on the button 300, fill and the button 400 empty the tank 100 and,
on the other hand, to allow the disassembly of said piston 120 to allow the removal of the reservoir 100 from the body 200.

This detachable fastening is implemented by a coupling module 500 in helical connection with the thrust shaft 221 which forms a worm screw. The coupling module 500 moves in forward or reverse translation (double arrow F1) when said screw is rotated in one direction or the other by the motor 220. A reduction module 222 is interposed between the shaft 221 and the motor 220, reducing the output speed and providing the necessary power to the shaft 221 and thus to the coupling module 500 to perform its functions.

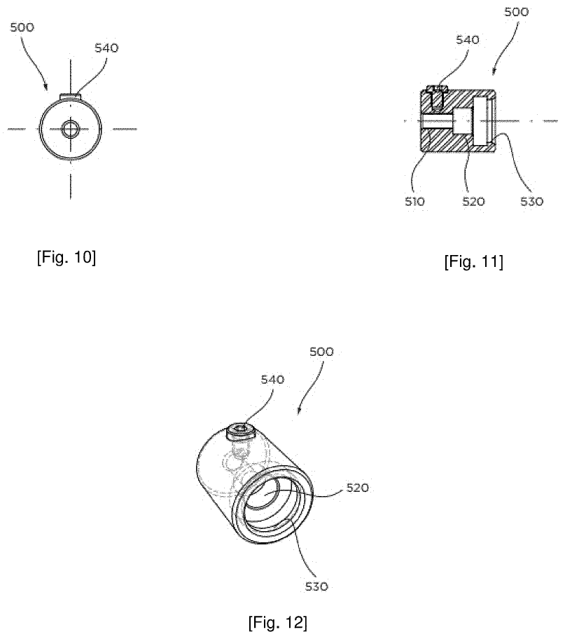

As illustrated in FIGS. 10, 11, and 12, this coupling module 500 includes an open hollow core partially occupied by an axial threaded portion 510 for helical connection to the shaft 221. This hollow core flares out and is preformed with a plurality of diameter changes including a bore 520 and a peripheral retaining rib 530 defining different inside diameters.

This bore 520 and rib 530 constitute fastening means for the tips that will be received by the device D.

Thus, the first attachment means 530 provides retention of the second end 132 of the rod 130 configured to set the rod 130 and thus the piston 120 in motion.

The second fastening means 520 cooperates with other pushing means which will be described below.

These first and second fastening means of the coupling module 500 are coaxial with the longitudinal axis of the thrust shaft 221.

The coupling module 500 is also equipped on its outer surface with at least one rotation-stopping projection 540 that prevents rotation by cooperating with a groove integral with the body 200. According to the illustrated embodiment, this projection 540 is implemented by the head of a screw screwed into the coupling module 500.

According to another non-illustrated embodiment, the coupling module is configured on its outer surface with at least one guide flat that, being in contact with a flat surface integral with the body, blocks the rotation.

This rotation lock ensures that the coupling module 500 has a translational motion during the rotation of the worm screw 221 with which it is in helical connection.

The first fastening means 530 thus serves as a retaining surface for the second end 132 of the piston rod 130, which, as illustrated in FIG. 8, is configured with a plurality of slots 133 as well as a retaining bead 134 in order to cooperate with the coupling module 500 by being fixed thereto by elastic deformation. Through the elastic deformation permitted by the slots 133, the end 132 can thus penetrate the hollow core of the coupling module 500 until the peripheral bead 134 redeploys (clipping) and is retained in position by the rib 530. Translation of the coupling module 500 (double arrow F1) can then be transmitted to the piston rod 130 for filling or emptying operations.

The second end 132 of the piston rod 130 further includes a peripheral disengagement wedge 135 provided to disengage the reservoir 100 from the body 200 of the device D.

At one end of the retraction movement of the piston 120 reached under the pushing action of the coupling module 500 after the emptying movement, this disengagement wedge 135 comes into contact with a disengagement stop 600 associated with the body 200 (see FIGS. 6, 7) allowing the end 132 to shrink radially in an elastic manner so that its retaining bead 134 is no longer retained by the coupling module 500.

The coupling module 500 includes a second fastening means constituted by the bore 520 allowing the immobilization by press fit of a second pushing means or shaft 700 which cooperates with a second series of connecting tips but which is not illustrated without interfering with the use of the first series of connection tips.

This second pushing means is in the form of a piston 710 with a rod 720 and a rear rod end 721 sized to form an interference fit with the bore 520 of the coupling module 500. The piston 710 is sized to slide within the hollow core 136 of the piston rod 130 of the tank 100.

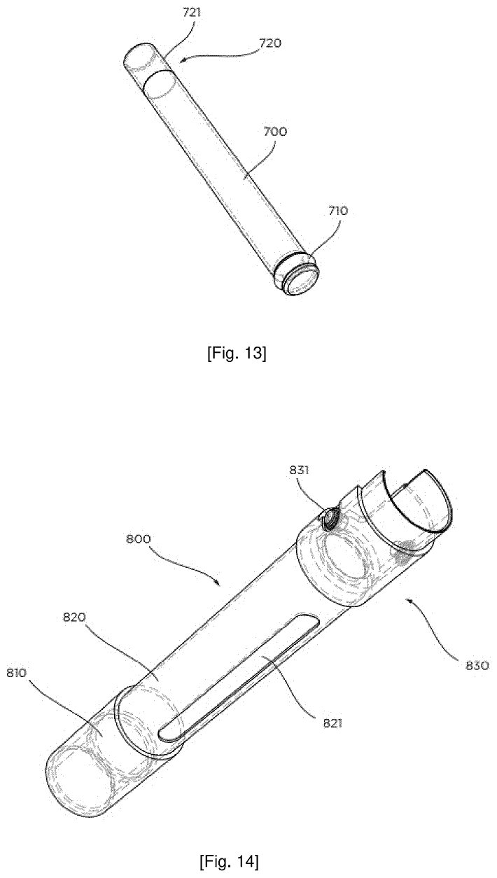

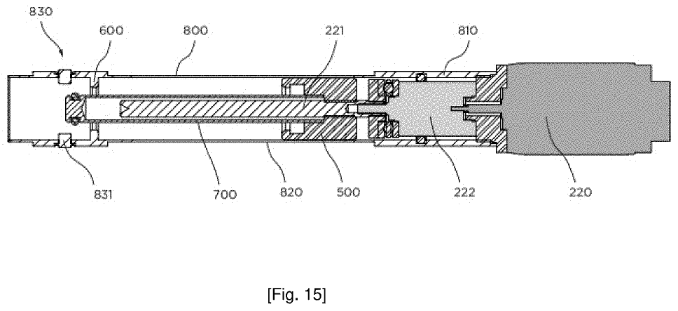

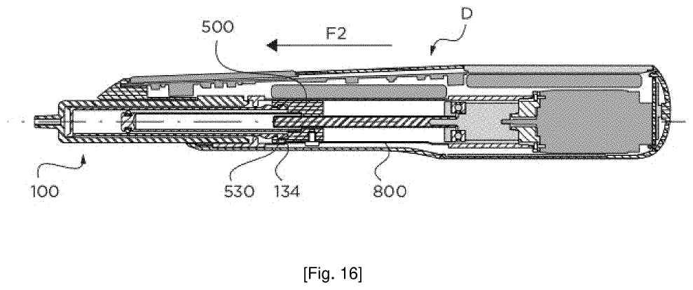

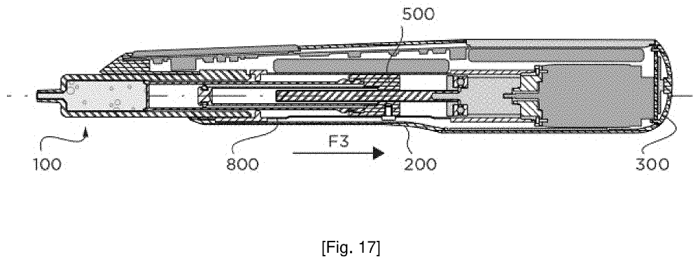

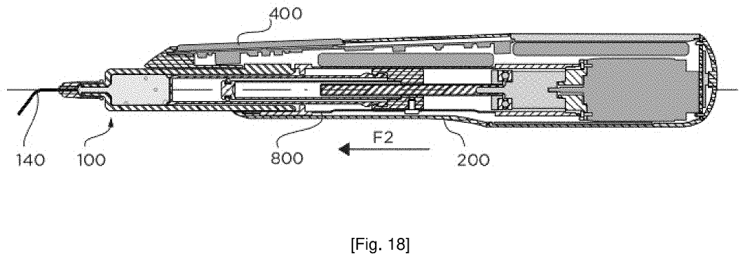

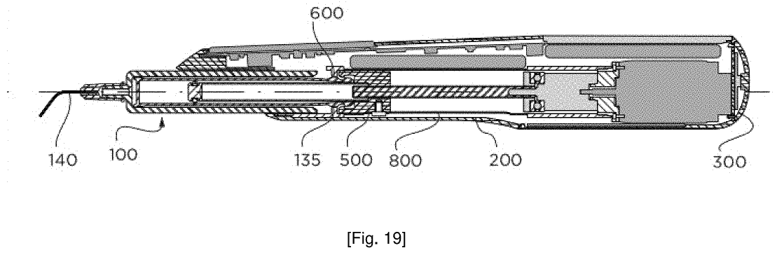

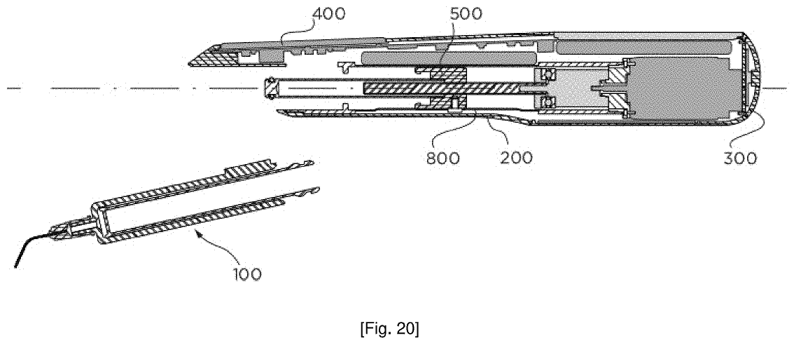

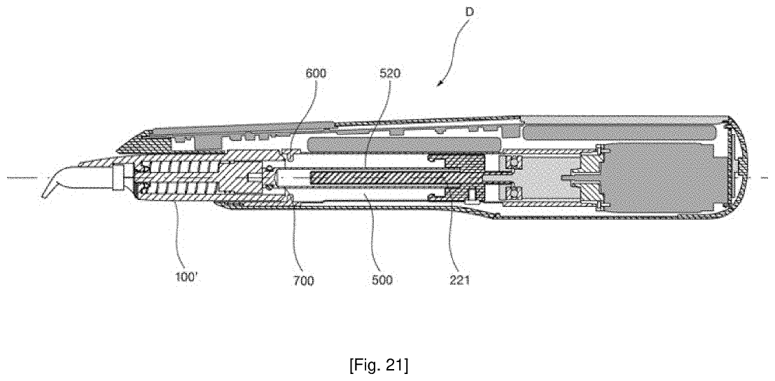

FIGS. 14 and 15 illustrate a motor support part 800 that participates by its shapes in a plurality of functions implemented by the device D.

Positioned within and attached to the body 200, the motor support 800 has a substantially hollow cylindrical shape open at both ends. A first open end 810 is configured to secure the motor 220.

A hollow middle portion 820 accommodates and guides the sliding of the coupling module 500. In order to block its rotation, a longitudinal groove 821 is made so as to cooperate with the projection 540 associated with the coupling module 500.

The second end 830 accommodates the connection tip or piston reservoir 100 and is notably configured with the above-mentioned disengagement stop 600 formed here by a peripheral rib.

According to another embodiment not shown, this peripheral rib is implemented by an elastic ring such as a circlip, positioned in a peripheral groove formed in the second end of the motor support.

This second end 830 is also equipped with two bayonets 831 projecting internally. These bayonets 831 serve as means of attachment for the tank 110 (see FIG. 8) which is formed with notches for this purpose.

This attachment is illustrated in particular by FIG. 6A.

The various functional elements having been described separately, an example of the use of the device D for filling/emptying a piston reservoir 100 such as a syringe is described below with respect to FIGS. 6, 7, 16 to 20.

The situation illustrated by FIGS. 6 and 7 corresponds to the one where the connection tip or piston reservoir 100 to be filled has just been introduced into the body 200 through the open end 210. The reservoir 110 is then brought into position by the internal surfaces of the body 200 and more precisely by those of the motor support 800 described above. It is held in position by position-holding means such as bayonets 831 cooperating with notches 111 made in the tank 110.

The user's action on the button 400 triggers the motor 220, which turns the worm screw 221 so that the coupling module 500, moving in translation according to arrow F2, comes forward and couples with the end 132 of the fixed rod 130. The peripheral rib 530 engages and retains the peripheral bead 134.

The action of the user on the button 300 allows him to make the coupling module 500 return (arrow F3) towards the rear and to create the vacuum inside the tank 110 necessary for its filling as illustrated by FIG. 17. The device 200 may be used as illustrated in FIG. 4.

The use illustrated by FIG. 18 includes emptying the filled tank and therefore of a pushing action according to arrow F2. This pushing action can be controlled by the button 400.

When the connection tip 100 is locked on the bayonets 831 (see FIG. 6A), the peripheral disengagement wedges 135 cannot reach the disengagement stop 600 because the end 131 is in abutment with the bottom of the tank 110. A rotation of the connection tip 100 thus leads to the disengagement of the reservoir 110 from the bayonets 831 thus allowing the disengagement or release of the reservoir 100 illustrated by FIG. 19 which consists of the continuation of the movement of the coupling module 500 in the direction of the arrow F2 so that the peripheral disengagement wedge 135 preformed in the second end 132 of the piston rod 130 comes into contact with the disengagement stop 600 preformed in the motor support 800 fixed to the body 200. The elasticity The elastic deformation of the end 132 then leads to its retaining bead 134 no longer being retained by the peripheral rib 530 of the coupling module 500.

As illustrated in FIG. 9, the end 132 is not only slotted (slots 133) but also equipped with a hollow core 136 that flares outwardly so that the tabs formed are reduced in thickness as they move away from their base. This thickness decrease created by a truncated conical inner surface 137 allows the tabs to have more angular flapping so as to facilitate disengagement especially when said tabs are likely to come in contact with the shaft 700.

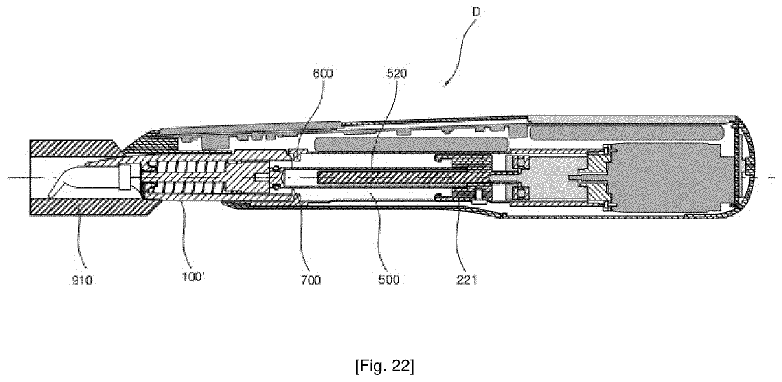

FIGS. 22, 23 and 24 illustrate different solutions envisaged for heating the connection tip and therefore its contents.

According to the embodiment illustrated by FIG. 22, the heating means is a heating ring 910 into which the connection tip 100′ positioned in the body 200 of the device D.

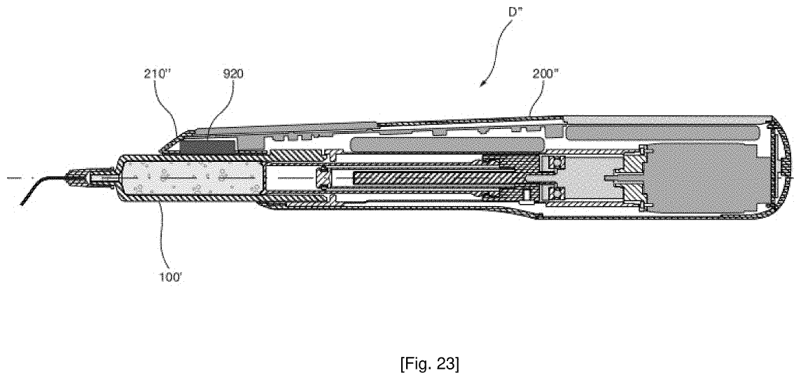

According to the embodiment illustrated in FIG. 23, the heating means 920 is integrated into the body 200″ of the device D″. This heating means 920 is integrated in the receiving volume of the tip defined in the open end 210″ and comes into contact with the tip when installed.

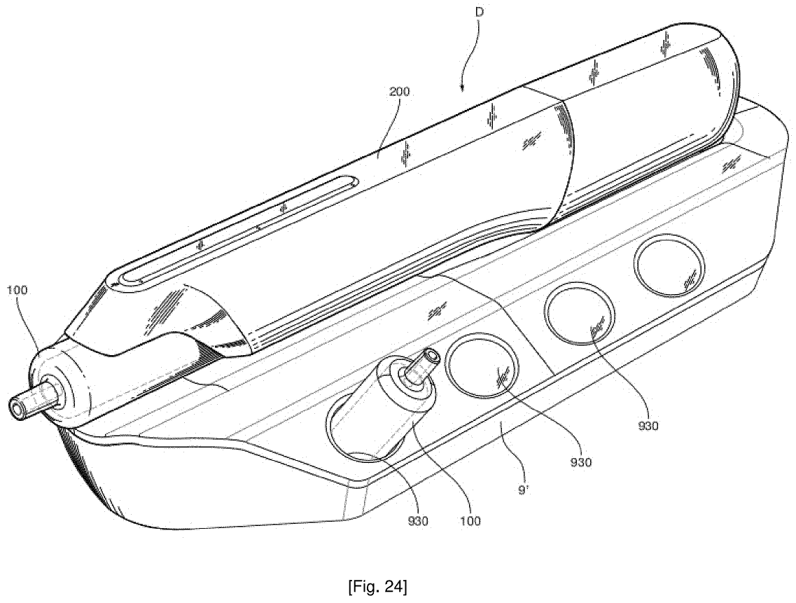

FIG. 24 illustrates an embodiment where the base S′ has a plurality of housings 930 for receiving the tips 100. One or more of these housings 930 comprises means for heating (not shown) the received end accommodated.

It is understood that the devices just described and represented above have been described with a view to disclosure rather than limitation. Of course, various adjustments, modifications and improvements may be made to the above examples, without going beyond the scope of the invention.

1. A portable medical device (D) for using an interchangeable connecting tip, said device comprising an elongated body carried and used with one hand,
said body being preformed at one end to removably receive said connecting tip
said connecting tip comprising a reservoir and a piston associated with a rod
which rod comprises a first and a second rod end, the first rod end being equipped with the piston moving in said reservoir
said device (D) ensuring the motorization of the movement of said piston,
the device comprising an actuator ensuring the movement of a first shaft acting on the second end of the rod
said actuator being controlled by the user by means of at least one control button,
WHEREIN
said tank is an empty tank to be filled and the first shaft is equipped to be temporarily fixed to the second end of the rod associated with the piston so as to, on the one hand, transmit by means of the first shaft on the one hand, to transmit, by means of the shaft, a forward and return translation movement (double arrow F1) to said piston so that the user can, by action on the control button , fill and empty said reservoir and, on the other hand, to authorize the disassembly of the rod in order to allow the disassembly of the connection tip from the body.

2. The Device (D) according to claim 1, WHEREIN a coupling module is in helical connection with the first thrust shaft formed by a worm screw in order to be moved in forward and return translation when said screw is driven in rotation, said coupling module including a first fastening means for retaining first thrust means for cooperation with a first series of connecting tips from a range of connection tips.

3. The device (D) according to claim 2, WHEREIN the coupling module comprises a second shaft which, providing a thrust only, is intended to cooperate with a second series of connecting pieces.

4. The device (D) of claim 3, WHEREIN the coupling module includes a second fastening means for attaching in a removable way the second push shaft.

5. The device (D) of claim 3, WHEREIN the first and second fastening means of the coupling module are coaxial with the longitudinal axis of the worm.

6. The device (D) of claim 2, WHEREIN the coupling module has a rotation locking means on its outer periphery so that its movement along the shaft is a translation.

7. The device (D) of claim 2, the second end of the rod associated with the piston is preformed with a retaining bead in order to cooperate with the coupling module.

8. The Device (D) of claim 2, WHEREIN the second end of the rod associated with the piston is preformed with a plurality of slots to cooperate with the coupling module by being fixed thereto by elastic deformation.

9. The Device (D) of claim 7, WHEREIN the second end of the rod associated with the piston comprises a peripheral disengagement wedge which engages at one end of the stroke of the coupling module with a disengagement stop allowing it to deform elastically so that its retaining bead is no longer retained in the coupling module.

10. The device (D) of claim 6, WHEREIN the second end of the rod associated with the piston is preformed from an outwardly flaring hollow core with a partially frusto-conical inner surface so that the lamellae created by the slots have a thickness that reduces from their base to their tip

11. The device (D) according to claim 1, WHEREIN the control button for the filling operations of the connection tip is disposed at the opposite end from that of the body of the device receiving the connection tip.

12. The device (D) of claim 1, WHEREIN one or more buttons participating in the use of the device (D) for operations other than filling are disposed closest to the first end receiving the tip.

13. The Device (D) according to claim 3, WHEREIN the coupling module in its translational stroke, couples to the second end of the rod associated with the piston once the connection tip is installed in the body in order to set it in motion in both translational directions.

14. The Device (D) according to claim 1, WHEREIN it comprises a base (S, S′) on which the body of the device can be positioned.

15. The Device (D, D″) according to claim 1, WHEREIN it comprises means for heating the tip

16. The device according to claim 15, WHEREIN the means for heating the tip is integrated or associated with one of the following sub-assemblies of the following sub-assemblies:
the body of the device,
the tip,
the support and recharge base.
